(19) United States
(12) Patent Application Publication
(10) Pub. No.: US 2026/0144786 A1
(43) Pub. Date: May 28, 2026

(54) METHOD FOR TREATING MENSTRUAL CYCLE DISORDERS

(71) Applicant: Guangzhou HuTong Youwu Biotechnology Co., Ltd, Guangzhou (CN)

(72) Inventors: Wei ZHU, Guangzhou (CN); Xiaowen ZHANG, Guangzhou (CN)

(73) Assignee: Guangzhou HuTong Youwu Biotechnology Co., Ltd, Guangzhou (CN)

(21) Appl. No.: 19/452,575

(22) Filed: Jan. 19, 2026

Related U.S. Application Data
(63) Continuation of application No. PCT/CN2024/106625, filed on Jul. 22, 2024.

(30) Foreign Application Priority Data
Jul. 24, 2023 (CN) 202310915716.3

(51) Int. Cl. A61K 31/4745; A61P 15/00

(52) U.S. Cl. CPC A61K 31/4745; A61P 15/00

(57) ABSTRACT

Disclosed is a method for treating a menstrual cycle disorder in a patient in need thereof, comprising: orally administering to the patient a therapeutically effective amount of pyrroloquinoline quinone (PQQ), or a pharmaceutically acceptable derivative thereof. Preliminary experimental data show that oral PQQ has a significant effect on preventing, alleviating, or treating menstrual cycle disorders compared with a placebo.

### CROSS-REFERENCE TO RELATED APPLICATIONS

The present application is a continuation-in-part application of PCT application No. PCT/CN2024/106625 filed on Jul. 22, 2024, which claims the benefit of Chinese Patent Application No. 202310915716.3 filed on Jul. 24, 2023. The contents of all of the aforementioned applications are incorporated by reference herein in their entirety.

### TECHNICAL FIELD

The present disclosure relates to a new use of a compound, specifically to the use of pyrroloquinoline quinone (PQQ) in the preparation of a modulator for menstrual cycle disorders.

### BACKGROUND OF THE INVENTION

The normal menstrual cycle of women is 21 to 35 days, with an average menstrual cycle of 28 days. The menstrual period is generally 2 to 8 days, with an average of 4 to 6 days. The menstrual volume is the total blood loss during one menstrual period. The normal menstrual volume is 20 to 60 ml, and more than 80 ml is considered menorrhagia.

Symptoms of menstrual disorders mainly include:

1. Early menstruation: Early menstruation refers to a shortened menstrual cycle that is shorter than 21 days and occurs for more than two consecutive cycles. It presents with a biphasic basal body temperature of ovulatory dysfunctional uterine bleeding, a short follicular phase of only 7 or 8 days; or a luteal phase of less than 10 days, or a temperature rise of less than 0.5° C.

2. Delayed menstruation: Menstruation is missed for more than 7 days, or even occurs every 40-50 days, and symptoms appear for more than 2 consecutive menstrual cycles. For those who ovulate, the basal body temperature is biphasic, but the follicular phase is long and the high-temperature phase is relatively low; for those who do not ovulate, the basal body temperature is monophasic.

3. Prolonged menstruation: The symptoms of this type of menstrual disorder are mainly a normal menstrual cycle but a prolonged menstrual period, lasting more than 7 days, or even up to 2 weeks before ending. Those with inflammation usually experience lower abdominal pain, which is aggravated during menstruation. Usually, the amount of leucorrhea is heavy, yellow or yellowish-white, thick, and odorous. Those with incomplete corpus luteum atrophy are also accompanied by heavy menstruation; those with prolonged endometrial repair still experience a small amount of persistent vaginal bleeding after the normal menstrual period.

4. Irregular menstruation: Menstruation is early or late, and the cycle is shorter than 21 days or longer than 35 days.

Menstrual cycle disorder is a common disease among women. If it persists for a long time, mild cases will accelerate facial aging, and severe cases will lead to serious gynecological diseases, causing inconvenience to women's normal lives and affecting their health.

In the prior art, menstrual cycle disorders are mainly treated with hormones. Hormone therapy has significant side effects, drug resistance, and risk of recurrence; therefore, only a small number of women with severe menstrual disorders adopt this treatment regimen. There are also reports of using Traditional Chinese Medicine to treat menstrual cycle disorders, which show certain efficacy.

PQQ is a tricarboxylic acid quinone compound, CAS No. 72909-34-3, with the IUPAC Name: 4,5-dioxo-1H-pyrrolo[2,3-f]quinoline-2,7,9-tricarboxylic acid. PQQ is widely present in various food ingredients. The content of PQQ in food ingredients is about 3.65-61 ng/g. In vegetables such as parsley and green peppers, fruits such as kiwi and papaya, drinks such as green tea and Oolong tea, and tofu that people often eat, the content of PQQ is all about 30 ng/g. Previous studies have shown that PQQ can stimulate the rapid growth of microbial, plant, animal, and human cells; scavenge excess free radicals in the body and protect the body from oxidative damage; and accelerate the oxidation of acetaldehyde to acetic acid to reduce the content of acetaldehyde in the body, thereby being expected to reduce the toxic damage to the liver caused by alcohol consumption. CN110870866A discloses the application of pyrroloquinoline quinone in the preparation of medicines for preventing and treating acute altitude sickness and acute altitude hypoxia injury; CN110151764A discloses that pyrroloquinoline quinone can reduce lipopolysaccharide-induced animal fibroblast and intestinal inflammatory responses; CN106265730A discloses the application of PQQ in reversing tumor multidrug resistance; CN105963297A discloses the application of pyrroloquinoline quinone in adjuvant therapy after chemotherapy; CN103191115A discloses the application of pyrroloquinoline quinone in the treatment and/or amelioration of diabetic foot.

### SUMMARY OF PRESENT INVENTION

No studies have shown that PQQ can be used to treat menstrual cycle disorders. An object of the present disclosure is to overcome at least one disadvantage of the prior art and provide the use of PQQ in the preparation of a modulator for menstrual cycle disorders.

The technical solution adopted by the present disclosure is:

A method for treating a menstrual cycle disorder in a patient in need thereof, comprising: orally administering to said patient a therapeutically effective amount of pyrroloquinoline quinone, or a pharmaceutically acceptable derivative thereof.

In some embodiments, said menstrual cycle disorder presents with one of the following symptoms: early menstruation; delayed menstruation; prolonged menstruation; and irregular menstruation.

In some embodiments, said menstrual cycle disorder does not include menstrual cycle disorders caused by endometriosis.

In some embodiments, the derivative of pyrroloquinoline quinone is a derivative capable of being digested or metabolized into pyrroloquinoline quinone in vivo.

In some embodiments, the derivative of pyrroloquinoline quinone is a derivative capable of being hydrolyzed into pyrroloquinoline quinone in the digestive tract.

In some embodiments, the derivative of pyrroloquinoline quinone is a pharmaceutically or food-acceptable salt, C2-C6 ester, amide, hydrate, crystal, co-crystal, or solvate.

In some embodiments, the derivative of pyrroloquinoline quinone is the disodium salt of pyrroloquinoline quinone.

In some embodiments, the therapeutically effective amount, calculated as pyrroloquinoline quinone, is: 5 to 60 mg/day for preventing menstrual cycle disorders; and 10 to 800 mg/day for ameliorating or treating menstrual cycle disorders.

In some embodiments, said pyrroloquinoline quinone is administered in the form of a pharmaceutical composition or a food product.

### BENEFICIAL EFFECTS

Preliminary experimental data show that oral PQQ has a significant effect on preventing, alleviating, or treating menstrual cycle disorders compared with a placebo.

### DETAILED DESCRIPTION OF THE EMBODIMENTS

In the present disclosure, the pharmaceutically or food-acceptable derivatives of the compound refer to simple derivatives thereof, especially one of the lower esters, lower ethers, lower alkyl substituents, pharmaceutically acceptable salts, and lower amides, which are derivatives obtained by condensation of a carboxylic acid, alcohol, or amine having 1 to 6 carbon atoms, preferably 2 to 6, or 2 to 4 carbon atoms, with the parent compound. In particular, the general formula of the derivative of pyrroloquinoline quinone is as follows:

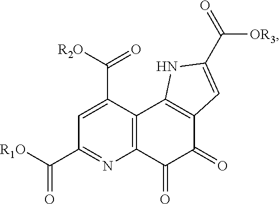

where, R₁ to R₃ are the same or different, and are selected from H, C1 to C6 hydrocarbon groups, natural amino acid residues, Na, K, Li, and other pharmaceutically acceptable groups.

Pharmaceutically acceptable salts of the compounds can be synthesized from parent compounds by conventional chemical methods, such as those described in Pharmaceutical Salts: Properties, Selection, and Use, P. Heinrich Stahl (Editor), Camille G. Wermuth (Editor), ISBN: 3-90639-026-8, Hardcover, 388 pages, August 2002. In general, these salts are prepared by the reaction of the free base and acid of the compound in water or an organic solvent or a mixture of the two; typically, non-aqueous media such as ether, ethyl acetate, ethanol, isopropanol, or acetonitrile are used.

Acid addition salts can be prepared using various acids (inorganic and organic acids). Examples of acid addition salts include salts formed with an acid selected from the group consisting of acetic acid acetic acid, 2,2-dichloroacetic acid, adipic acid, alginic acid, ascorbic acid, L-aspartic acid, benzenesulfonic acid, benzoic acid, 4-acetylamino benzoic acid, butyric acid, (+)-camphoric acid, camphor sulfonic acid, (+)-(1S)-camphor-10-sulfonic acid, capric acid, hexanoic acid, octanoic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactonic acid, gentisic acid, glucoheptonic acid, D-gluconic acid, glucuronic acid, glutamic acid, α-ketoglutaric acid, glycolic acid, hippuric acid, hydrobromic acid, hydrochloric acid, hydroiodic acid, isethionic acid, (+)-L-lactic acid, (±)-DL-lactic acid, lactobionic acid, maleic acid, malic acid, (−)-L-malic acid, malonic acid, (±)-DL-mandelic acid, methanesulfonic acid, naphthalene-2-sulfonic acid, naphthalene-1,5-disulfonic acid, 1-hydroxyl-2-naphthoic acid, nicotinic acid, nitric acid, oleic acid, orotic acid, oxalic acid, palmitic acid, pamoic acid, phosphoric acid, propionic acid, L-pyroglutamic acid, salicylic acid, 4-aminosalicylic acid, sebacic acid, stearic acid, succinic acid, sulfuric acid, tannic acid, (+)-L-tartaric acid, sulfocyanic acid, p-toluenesulfonic acid, undecylenic acid, pentanoic acid, and acylated amino acid and acylated amino acids and cation exchange resins.

The technical solution of the present disclosure is further explained below in conjunction with experiments.

The criteria for menstrual cycle disorders are as follows:

1. Early menstruation: Early menstruation refers to a shortened menstrual cycle that is shorter than 21 days and occurs for more than two consecutive cycles. It is a biphasic basal body temperature of ovulatory functional uterine bleeding, a short follicular phase of only 7 or 8 days; or a luteal phase of less than 10 days, or a temperature rise of less than 0.5° C.

2. Delayed menstruation: Menstruation is missed for more than 7 days, or even 40-50 days, and symptoms appear for more than 2 menstrual cycles. For those who ovulate, the basal body temperature is biphasic, but the follicular phase is long and the high-temperature phase is low; for those who do not ovulate, the basal body temperature is monophasic.

3. Prolonged menstruation: The symptoms of this type of menstrual disorder are mainly a normal menstrual cycle but a prolonged menstrual period, lasting more than 7 days, or even up to 2 weeks before ending. Those with inflammation usually experience lower abdominal pain, which is aggravated during menstruation. Usually, the amount of leucorrhea is heavy, yellow or yellowish-white, thick, and odorous. Those with incomplete corpus luteum atrophy are also accompanied by heavy menstruation; those with prolonged endometrial repair still experience a small amount of persistent vaginal bleeding after the normal menstrual period.

4. Irregular menstruation: Menstruation is early or late, and the cycle is shorter than 21 days or longer than 35 days.

No endometriosis.

### Use of PQQ for Preventing Menstrual Cycle Disorders

The selection criteria were the presence of one menstrual cycle disorder within 6 menstrual cycles, age between 16 and 30 years old. The prevention method was oral administration of tablets containing 8 mg PQQ once daily. The experiment lasted for six months. The control group took a tablet containing the same amount of vitamin C orally once a day.

### Use of PQQ for Ameliorating or Treating Menstrual Cycle Disorders

The selection criteria were the presence of at least 3 menstrual cycle disorders within 6 menstrual cycles, age between 18 and 40 years old. During the treatment period, tablets containing 50 mg PQQ were taken orally once daily. The experiment lasted for six months. The control group took a tablet containing the same amount of vitamin C orally once a day.

The experimental results are shown in Table 1.

TABLE 1
Experimental results

| | Number of | Number of | | |
|---|---|---|---|---|
| | subjects in | subjects in | | Efficacy in |
| | experimental | control | Efficacy in | experimental |
| Group | group | group | control group | group |
| Prevention group | 50 | 50 | No significant | Significant |
| | | | improvement | improvement |
| Early menstruation | 40 | 40 | No significant | Significant |
| (shortened menstrual | | | improvement | improvement |
| cycle) | | | | |
| Delayed menstruation | 50 | 50 | No significant | Significant |
| | | | improvement | improvement |
| Prolonged menstruation | 50 | 50 | No significant | Significant |
| | | | improvement | improvement |
| Irregular menstruation | 35 | 35 | No significant | Significant |
| | | | improvement | improvement |

The above is a further detailed description of the present disclosure, which should not be regarded as a limitation on the specific implementation of the present disclosure. For those of ordinary skill in the art to which the present disclosure belongs, simple deductions or replacements without departing from the concept of the present disclosure are all within the protection scope of the present disclosure.

What is claimed is:

1. A method for treating a menstrual cycle disorder in a patient in need thereof, comprising: orally administering to the patient a therapeutically effective amount of pyrroloquinoline quinone, or a pharmaceutically acceptable derivative thereof.

2. The method according to claim 1, wherein the therapeutically effective amount, calculated as pyrroloquinoline quinone, is:
5 to 60 mg/day for preventing menstrual cycle disorders;
o 800 mg/day for ameliorating or treating menstrual cycle disorders.

3. The method according to claim 1, wherein the menstrual cycle disorder presents with one of the following symptoms:
early menstruation;
delayed menstruation;
prolonged menstruation;
irregular menstruation.

4. The method according to claim 1, wherein said menstrual cycle disorder does not include menstrual cycle disorders caused by endometriosis.

5. The method according to claim 1, wherein the pharmaceutically acceptable derivative of pyrroloquinoline quinone is a derivative capable of being digested or metabolized into pyrroloquinoline quinone in vivo.

6. The method according to claim 5, wherein the pharmaceutically acceptable derivative of pyrroloquinoline quinone is a derivative capable of being hydrolyzed into pyrroloquinoline quinone in the digestive tract.

7. The method according to claim 5, wherein the pharmaceutically acceptable derivative of pyrroloquinoline quinone is a pharmaceutically or food-acceptable salt, C2-C6 ester, amide, hydrate, crystal, co-crystal, or solvate.

8. The method according to claim 1, wherein the pyrroloquinoline quinone is administered in form of a pharmaceutical composition or a food product.
